(19) United States
(12) Patent Application Publication
(10) Pub. No.: US 2017/0258620 A1
(43) Pub. Date: Sep. 14, 2017

(54) PODIATRIC PRESSURE UNLOADING DEVICE

(71) Applicant: JASON HAMMOND, MD, LLC, REISTERSTOWN, MD (US)

(72) Inventor: Jason Hammond, REISTERSTOWN, MD (US)

(21) Appl. No.: 15/455,682

(22) Filed: Mar. 10, 2017

Related U.S. Application Data
(60) Provisional application No. 62/306,423, filed on Mar. 10, 2016.

(51) Int. Cl. A61F 5/01; A43B 7/00; A61F 5/14

(52) U.S. Cl. CPC A61F 5/0111; A61F 5/14; A43B 7/00

(57) ABSTRACT

A pressure unloading device for unloading pressure on a pathological prominence of the foot includes a securement and a spacer. The securement wraps around a portion of a foot and secures a spacer to a region of the foot proximate to a pathological prominence in order to unload pressure on the prominence. Pressure unloading devices can further include footwear, such as a shoe or other footwear or footwear liner having voids and/or spacers.

### CROSS-REFERENCES TO RELATED APPLICATIONS

This application claims the benefit of U.S. Provisional Application No. 62/306,423, filed Mar. 10, 2016, the entire contents of which are hereby incorporated in its entirety for all purposes.

### BACKGROUND

Various painful musculoskeletal deformities affect the foot. Some deformities can impede comfort, shoe selection, and even proper biomechanics. For example, a bunion, or hallux abudcto valgus, is a deformity of the joint connecting the big toe to the foot. A bunion is generally characterized by medial deviation of the first metatarsal bone and lateral deviation of the hallux or big toe. The big toe points toward the other toes, and the base of the big toe is pushed outward away from the foot. The bunion presents as a bony prominence projecting laterally from the base of the big toe, which includes the deviated joint, as well as displaced and/or inflamed tissue in and around the joint forming a painful prominence.

Other podiatric deformities can result in similarly painful prominences. For example, a Taylor's bunion, or bunionette, is a condition caused by a similar deviation in the fifth metatarsal bone. The small toe deviates or angles inward toward the other toes, and the fifth metatarsal bone projects outward. Like a bunion, a Taylor's bunion results in a painful protrusion, but from the outside of the foot.

A dorsal midfoot prominence, caused by dorsal midfoot exostosis, is yet another potentially painful condition. Dorsal midfoot exostosis is characterized by a bony growth on top of the arch at the metatarso-cuneiform joint. The prominence, while often not directly painful, can rub against the shoe and become compressed, irritated, or inflamed. Irritation can also cause aggravation of the nerves in the top of the foot.

An accessory navicular prominence is caused by an accessory navicular bone, which is an accessory bone of the foot. An accessory navicular bone occasionally develops in front of the ankle along the side of the foot. While usually asymptomatic, in some cases, the accessory navicular bone can exacerbate plantar fasciitis, bunions, heel spurs, and irritation or compression of the tissue surrounding the bone.

A pathological prominence can also develop at the base of the fifth metatarsal. For example, injury to this region can often cause inflammation. Due to the presence of a large tendon, injuries to this region can take a long time to heal, and painful inflammation may linger, or be exacerbated by pressure.

Various other prominences, by virtue of being on the foot, can readily become injured and inflamed, thus restricting comfort and mobility in a similar manner. For example, the medial malleolus is the large, bony prominence on the inner side of the angle. The prominence can be injured by acute trauma, or by repetitive trauma, e.g., by repeated impact with footwear. In the event that inflammation develops, further impact or pressure on the prominence can exacerbate the injury. A ganglion, or ganglion cyst, is a common tumor or swelling on top of a joint or tendon. Although ganglions are most common on the hands, they can also occur on the top of the foot, where they are prone to injury. A “pump bump,” also called a Haglund's deformity, is a bony prominence on the back of the heel. The “pump bump” is characterized by irritation and enlargement of the tissue around the connective site of the Achilles tendon caused by, e.g., rigid shoes. Once inflamed, the deformity can be difficult to treat while retaining mobility.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

According to various embodiments, a prominence unloader includes a securement shaped to wrap around a part of a foot, and a spacer connected with the securement and configured to rest proximal or distal to a pathological prominence of the foot. The prominence has a first height, and the spacer has a second height that is substantially equal to or greater than the first height. The securement is arranged to wrap around the foot such that it secures the spacer on a surface of the foot proximate to the pathological prominence. Generally, the securement secures the spacer on a surface of the foot adjacent or near the prominence without resting on the prominence. In some cases, the securement secures the spacer abutting the prominence, partially around the prominence, fully around the prominence, and/or partially on top of the prominence.

In some embodiments, a prominence unloader includes one or more secondary securement straps. Secondary securement straps can prevent rotation of the securement around the foot. In some cases, securement straps may extend from a primary securement to an ankle strap or from a primary securement to a toe strap. In some other cases, securement straps may wrap around another part of a foot.

Various embodiments of a prominence unloader may be arranged to accommodate particular pathological prominences. For example, a securement of a prominence unloader may be arranged to secure the spacer proximate to a bunion, a Taylor's bunion, a medial malleolus, a ganglion, a “pump bump,” a lateral malleolus, or other suitable pathological prominence.

According to various embodiments, a brace can be arranged to wrap around a portion of a foot and provide support to the foot, while also accommodating a spacer for unloading pressure on a pathological prominence. The spacer may be integrally connected with the brace and configured to rest proximal to a pathological prominence of the foot when the brace is worn. Where the prominence has a first height, the spacer has a second height that is generally equal to or greater than the first height. The brace is configured to secure the spacer at a surface of the foot proximate to the pathological prominence.

According to various embodiments, a shoe can be configured to provide a spacer integrally connected with the shoe for unloading a pathological prominence of a foot. The spacer is arranged in the shoe to rest proximal or distal to the pathological prominence of the foot when the shoe is worn. Where the prominence has a first height, the spacer has a second height that is generally equal to or greater than the first height. In some embodiments, the shoe also has a void adjacent to the spacer and arranged to fit around the prominence, so as to unload pressure on the prominence.

According to various embodiments, a boot liner, e.g. for a sport shoe or sport boot like a ski boot, hiking boot, or the like, can be configured to provide for unloading of a pathological, bony prominence in a foot. For example, one or multiple pockets may be provided in a boot liner that can receive spacers to unload a bony prominence that is laterally adjacent to the pocket or pockets. By way of another example, one or multiple pockets may be provided in a boot liner that are positioned directly adjacent (i.e., on top of) positions of one or more bony prominences, so that one or more spacers filling the pocket or pockets can be removed in order to unload the bony prominence.

For a fuller understanding of the nature and advantages of the present invention, reference should be made to the ensuing detailed description and accompanying drawings.

### BRIEF DESCRIPTION OF THE DRAWINGS

FIG. 1 illustrates an unloader assembly for unloading pressure on a bunion on a skeletal schematic of a foot, in accordance with embodiments;

FIG. 2 illustrates a second unloader assembly for unloading pressure on a bunion, having additional securement features;

FIG. 3 illustrates a third unloader assembly for unloading pressure on a bunion including an integrated therapeutic sock;

FIG. 4 illustrates an unloader assembly for unloading pressure on a Taylor's bunion;

FIG. 5 illustrates an unloader assembly for unloading pressure on a dorsal midfoot prominence;

FIG. 6 illustrates a second unloader assembly for unloading pressure on a dorsal midfoot prominence;

FIG. 7 illustrates a third unloader assembly for unloading pressure on a dorsal midfoot prominence;

FIG. 8 illustrates an unloader assembly for unloading pressure on an accessory navicular prominence;

FIG. 9 illustrates an unloader assembly for unloading pressure on a pathological prominence at the base of the fifth metatarsal;

FIG. 10 illustrates an unloader assembly for unloading pressure on a medial malleolus;

FIG. 11 illustrates an unloader assembly for unloading pressure on a ganglion;

FIG. 12 illustrates an unloader assembly for unloading pressure on a “pump bump”;

FIG. 13 illustrates an alternative unloader assembly for supporting a ball of a foot and unloading pressure on a bunion;

FIG. 14 illustrates an unloader assembly for unloading pressure on a medial malleolus and bracing a foot;

FIG. 15 illustrates an unloader assembly for unloading pressure on a dorsal midfoot prominence and bracing a foot;

FIG. 16 illustrates an unloader assembly for unloading pressure on an accessory navicular and bracing a foot;

FIG. 17 shows a side section view of a first layered assembly for a bunion unloader;

FIG. 18 shows a side section view of a second layered assembly for a pressure unloader;

FIG. 19 shows a side section view of a third layered assembly for a pressure unloader;

FIG. 20 shows a side section view of a fourth layered assembly for a pressure unloader;

FIG. 21 shows a perspective view of a pressure unloading shoe for bunions;

FIG. 22 shows a side section view of the pressure unloading shoe of FIG. 21;

FIG. 23 shows a side view of a pressure unloading boot liner;

FIG. 24 shows the pressure unloading boot liner of FIG. 23 in a rear view;

FIG. 25 shows a side view of a second pressure unloading boot liner; and

FIG. 26 shows a side view of a sports boot receiving the pressure unloading boot liner of FIG. 25.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

Embodiments herein described are directed to unloader assemblies for unloading pressure from a pathological prominence of a foot. According to several embodiments, an unloader assembly includes a spacer and a primary securement. In general, a primary securement includes a flexible and elastic band, strap, or sleeve that is shaped to wrap around a portion of a foot and remain stationary. The primary securement holds a spacer or spacers in place on a targeted portion of a patient's foot. Generally, spacers are positioned laterally adjacent the targeted portion of the foot, i.e. next to the targeted portion rather than directly on top of the targeted portion. For example, a spacer or set of spacers used to treat a bunion may be placed next to the bunion in any direction or combination of directions, while not placing pressure directly on the bunion. Various specific embodiments of spacers and primary securements are described below, with reference to the drawings.

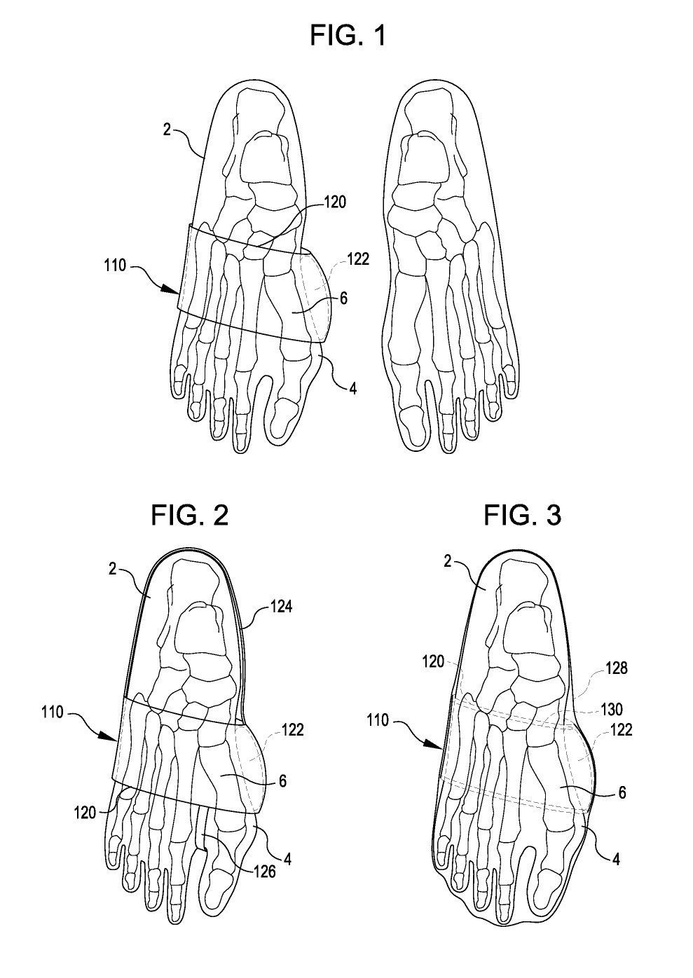

Referring now to the drawings, in which like reference numerals represent like parts throughout the several views, FIG. 1 shows a an unloader assembly 110 for unloading pressure on a bunion 4 on a foot 2, in accordance with embodiments. The unloader assembly 110 is arranged to project away from the foot 2 in order to unload pressure on bunion 4 from a surface, such as a shoe interior. The unloader assembly 110 has a primary securement 120 configured for wrapping around the foot 2 at the middle of the foot and securing a spacer 122 along the side of the foot, adjacent to the first metatarsal 6. The primary securement 120 can be flexible and elastic, so that it firmly supports the spacer 122. The spacer 122 is positioned laterally adjacent to a bunion 4 along the foot, and is arranged to project outward from the foot 2 approximately as much as, or more than, the bunion 4.

FIG. 2 shows another unloader assembly 110 for unloading pressure on a bunion 4 on a foot 2, in accordance with embodiments. The unloader assembly 110 is arranged like the unloader assembly 110 of FIG. 1, with the addition of supplemental securement features arranged to retain the position of the spacer 122 on the foot 2, and to prevent rotation and translation of the primary securement 120. For example, a heel strap 124 connects with the primary securement 120 and runs around a heel of the foot 2. A toe strap 126 connects with the primary securement 120 and runs between toes of the foot 2. Various embodiments of an unloader assembly 110 may possess one or both of a toe strap 126 and/or heel strap 124, or other suitable secondary securement features.

FIG. 3 shows another unloader assembly 110 for unloading pressure on a bunion 4 on a foot 2, in accordance with embodiments. The unloader assembly 110 is arranged like the unloader assembly 110 of FIG. 1, with the addition of a therapeutic sock 128. The therapeutic sock 128 is connected with, or integrally formed with, the primary securement 120. In some cases, the primary securement 120 can include a band of an elastic material that is attached to the sock 128, e.g., sewn, adhered, or integrally formed into the sock. In some cases, the primary securement 120 may be a region of the sock 128. In some other cases, the primary securement 120 may be formed into a cavity 130 in the material of the sock 128.

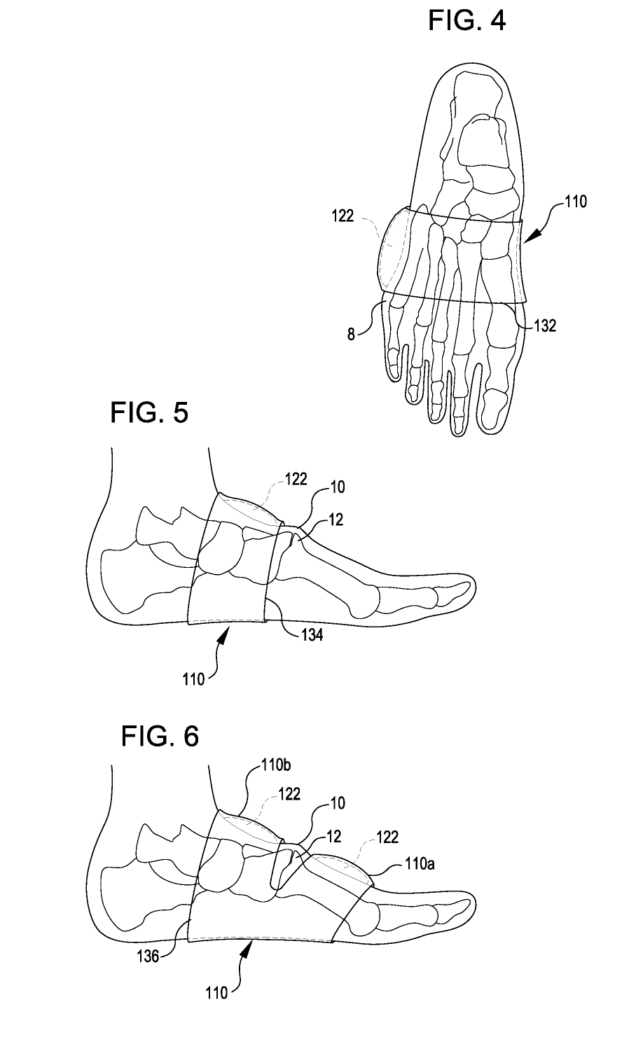

FIG. 4 shows an alternative unloader assembly 110 for unloading pressure on a Taylor's bunion 8 on a foot 2, in accordance with embodiments. The unloader assembly 110 is arranged to project away from the foot 2 in order to unload pressure on a Taylor's bunion from another surface, such as a shoe interior. The unloader assembly 110 has a primary securement 132 shaped to wrap around the middle of a foot 2 and secure the spacer 122 along the outer side of the foot 2 near the Taylor's bunion 8. The spacer 122 is adjacent to the Taylor's bunion 8 without pressing on the bunion, and is arranged to project outward from the foot 2 approximately as much, or more than, the Taylor's bunion.

FIG. 5 shows an alternative unloader assembly 110 for unloading pressure on a dorsal midfoot prominence 10, e.g., a prominence formed by dorsal midfoot exostosis 12 in accordance with embodiments. The unloader assembly 110 has a primary securement 134 arranged to wrap around the middle of the foot 2, proximal to the prominence 10. The primary securement 134 is further arranged to secure a spacer 122 adjacent and proximal to the dorsal midfoot prominence 10 along the top of the foot 2. The spacer 122 projects dorsally from the foot 2 adjacent to the dorsal midfoot prominence 10 approximately as much, or more than, the dorsal midfoot prominence.

FIG. 6 shows a second alternative unloader assembly 110 for unloading pressure on a dorsal midfoot prominence 10, e.g., a prominence formed by dorsal midfoot exostosis 12 in accordance with embodiments. The unloader assembly 110 includes a primary securement 136 that splits along the dorsal portion of the foot 2 into a distal part 110a and a proximal part 110b, each part supporting spacers 122. The spacers 122 are thus arranged both distal and proximal to the dorsal midfoot prominence 10. The spacers 122 project dorsally from the foot 2 approximately as much, or more than, the dorsal midfoot prominence 10.

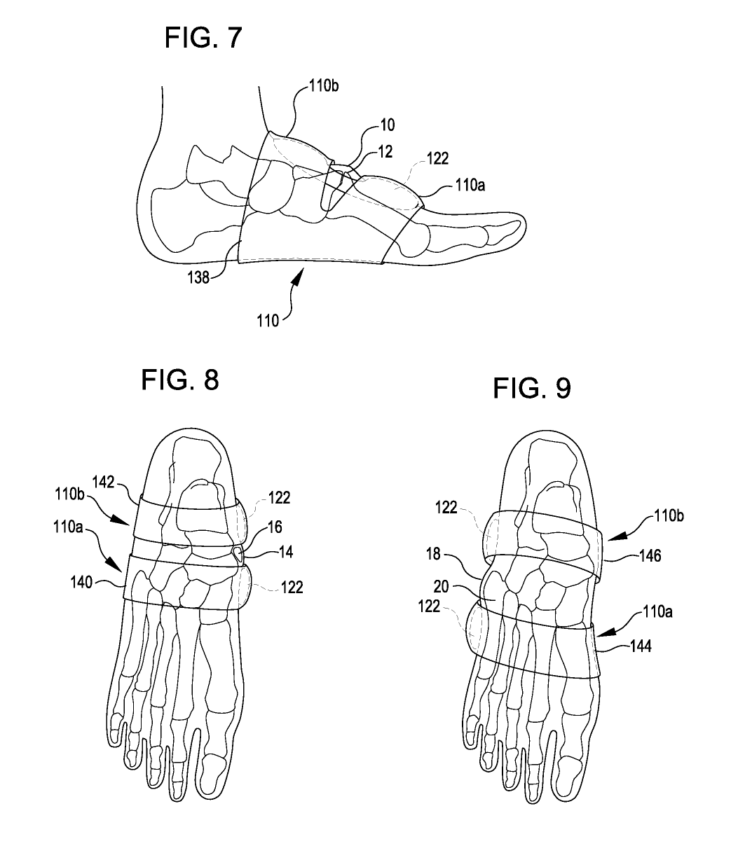

FIG. 7 shows a third alternative unloader assembly 110 for unloading pressure on a dorsal midfoot prominence 10, e.g., a prominence formed by dorsal midfoot exostosis 12 in accordance with embodiments. The unloader assembly 110 includes a primary securement 138 that splits along the dorsal portion of the foot 2 into a distal part 110a and a proximal part 110b. Each of the distal and proximal parts 110a, 110b supports a part of a spacer 122. The spacer 122 is arranged to wrap around the dorsal midfoot prominence 10. At least a portion of the spacer 122 projects dorsally from the foot 2 approximately as much, or more than, the dorsal midfoot prominence 10.

FIG. 8 shows an alternative unloader assembly 110 having a distal part 110a and proximal part 110b for unloading pressure on an accessory navicular prominence 14 caused by, e.g., a symptomatic accessory navicular 16, in accordance with embodiments. The unloader assembly 110 includes a distal securement 140 and a proximal securement 142, each securement arranged to wrap around a middle of the foot 2 distal to and proximal to the accessory navicular prominence 14, respectively. In some cases, the distal part 110a may be provided alone, or the proximal part 110b may be provided alone. The distal securement 140 and proximal securement 142 are each arranged to wrap the foot 2 and retain a spacer 122 adjacent to the accessory navicular prominence 14. The distal securement 140 secures a spacer 122 along the side of the foot 2 adjacent and distal to the accessory navicular prominence 14; and the proximal securement 142 secures another spacer 122 along the side of the foot adjacent and proximal to the prominence. Each spacer 122 projects outward from the side of the foot 2 approximately as much, or more than, the accessory navicular prominence 14.

FIG. 9 shows an alternative unloader assembly 110 having a distal part 110a and proximal part 110b for unloading pressure on a pathological prominence 18 of the base of the fifth metatarsal 20, in accordance with embodiments. The unloader assembly 110 includes a distal securement 144 and a proximal securement 146, each securement arranged to wrap around a middle of the foot 2 distal to and proximal to the pathological prominence 18, respectively. In some cases, the distal part 110a may be provided alone, or the proximal part 110b may be provided alone. The distal securement 144 and proximal securement 146 are each arranged to wrap the foot 2 and retain a spacer 122 adjacent to the pathological prominence 18. The distal securement 144 secures a spacer 122 along the side of the foot 2 adjacent and distal to the pathological prominence 18; and the proximal securement 146 secures another spacer 122 along the side of the foot adjacent and proximal to the prominence. Each spacer 122 projects outward from the side of the foot 2 approximately as much, or more than, the pathological prominence 18.

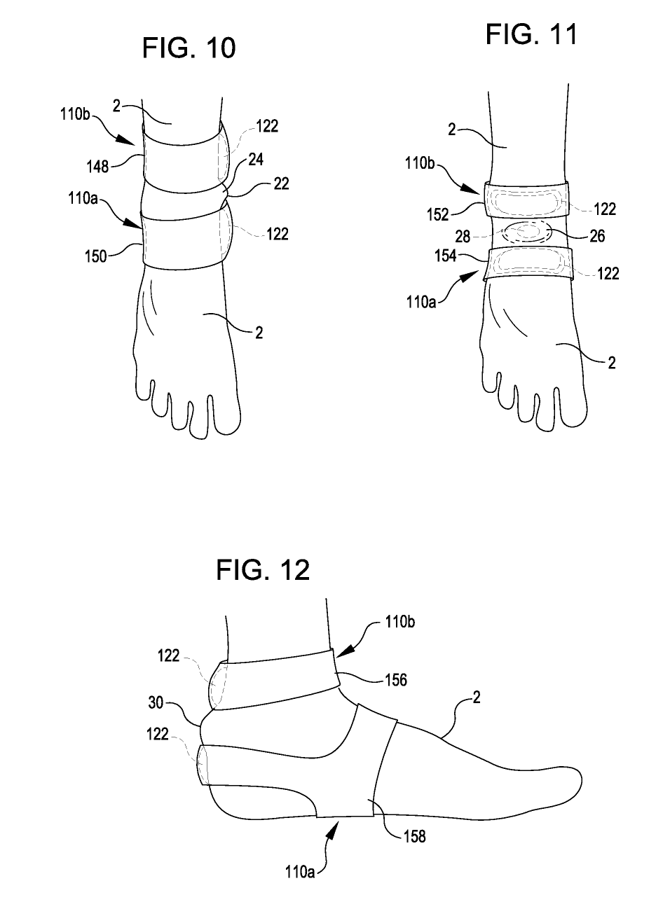

FIG. 10 shows an alternative unloader assembly 110 having a lower part 110a and an upper part 110b for unloading pressure on a medial malleolus along an inner, lateral portion of the ankle or foot 2, in accordance with embodiments. The unloader assembly 110 includes a lower securement 150 and an upper securement 148. In some cases, the lower part 110a may be provided alone, or the upper part 110b may be provided alone. The lower part 110a includes a lower securement 150 which wraps around a lower portion of the foot 2 and secures a spacer 122 adjacent to and below the medial malleolus 30 at the inner, lateral part of the foot 2. The upper part 110b includes an upper securement 148 which wraps around an upper part of the foot 2 and/or ankle and supports a spacer 122 adjacent to and above the medial malleolus 30. Each spacer 122 projects outward from the foot 2 approximately as much, or more than, the medial malleolus 30.

FIG. 11 shows an alternative unloader assembly 110 having a lower part 110a and an upper part 110b for unloading pressure on a ganglion 26 above a cyst 28 on the dorsal part of the foot 2, in accordance with embodiments. The unloader assembly 110 includes a lower securement 154 and an upper securement 152. In some cases, the lower part 110a may be provided alone, or the upper part 110b may be provided alone. The lower part 110a includes a lower securement 154 which wraps around a lower portion of the foot 2 and secures a spacer 122 adjacent to and distal to the ganglion 26 at the dorsal part of the foot 2. The upper part 110b includes an upper securement 152 which wraps around an upper part of the foot 2 and/or ankle and supports a spacer 122 adjacent to and proximal to the ganglion 26. Each spacer 122 projects dorsally from the foot 2 approximately as much, or more than, the ganglion 26.

FIG. 12 shows an alternative unloader assembly 110 having a lower part 110a and an upper part 110b for unloading pressure on a “pump bump” along a posterior end of the foot 2, in accordance with embodiments. The unloader assembly 110 includes a lower securement 158 and an upper securement 156. In some cases, the lower part 110a may be provided alone, or the upper part 110b may be provided alone. The lower part 110a includes a lower securement 158 which wraps around a lower portion of the foot 2 and secures a spacer 122 adjacent to and below the “pump bump” 30 at the posterior end of the foot 2. The upper part 110b includes an upper securement 156 which wraps around an upper part of the foot 2 and/or ankle and supports a spacer 122 adjacent to and above the “pump bump” 30. Each spacer 122 projects posteriorly from the foot 2 approximately as much, or more than, the “pump bump” 30.

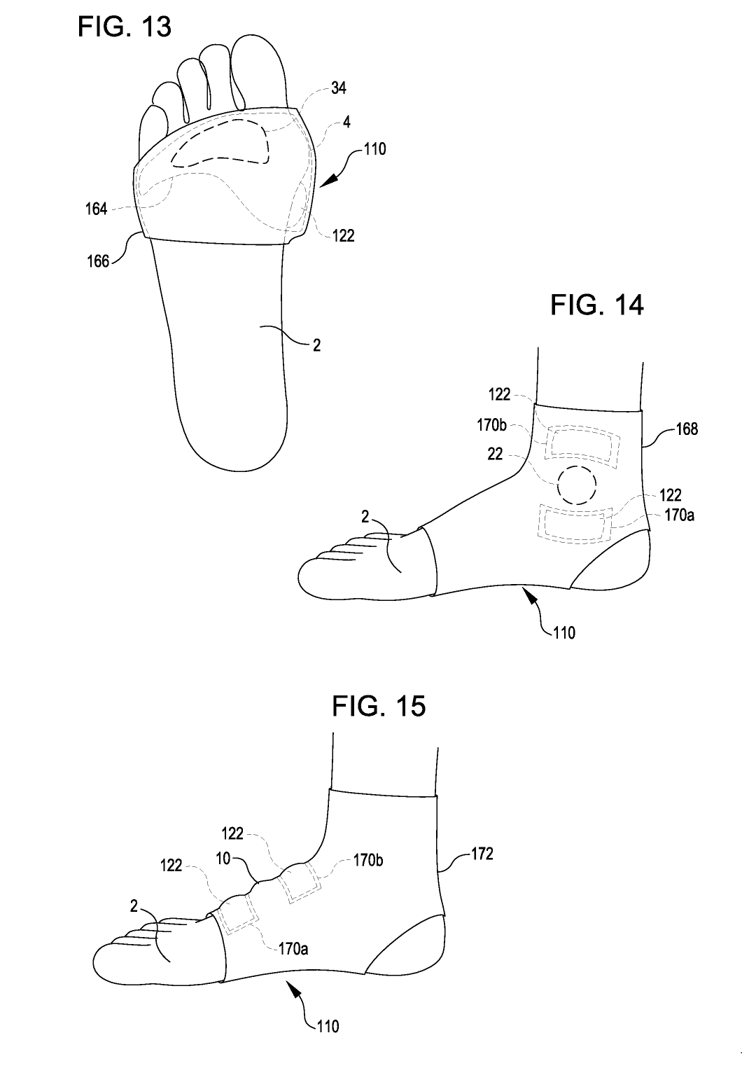

FIG. 13 shows an alternative unloader assembly 110 for unloading pressure on a bunion 4 and supporting the ball 34 of the foot 2, in accordance with embodiments. The unloader assembly 110 is arranged to project away from the foot 2 in order to unload pressure on the bunion 4 from a surface, such as a shoe interior. The unloader assembly 110 has a primary securement 166 configured for wrapping around the foot 2 at the middle of the foot and securing a spacer 122 along the side of the foot, proximal to the bunion 4. The primary securement 166 can also include a support 164 for cushioning and/or supporting the ball 34 of the foot 2. The spacer 122 is positioned adjacent to a bunion 4, and is arranged to project outward from the foot 2 approximately as much as, or more than, the bunion 4.

FIG. 14 shows an alternative unloader assembly 110 for unloading pressure on a medial malleolus 22 and bracing the foot 2 at the ankle, in accordance with embodiments. The unloader assembly 110 includes a brace 168 formed of a flexible, elastic material that wraps around the foot 2 where it joins the ankle. The brace 168 can provide mechanical support to the foot 2 and ankle. The brace 168 has a lower pocket 170a and an upper pocket 170b arranged below and above the medial malleolus 22, respectively. The lower and upper pockets 170a, 170b retain spacers 122. The spacers 122 project outward from the foot 2 above and below the medial malleolus 22. In some embodiments, the ankle brace 168 may include one of the lower and upper pockets 170a or 170b, rather than both. In some embodiments, one or both of the spacers 122 may be removable.

FIG. 15 shows an alternative unloader assembly 110 for unloading pressure on a dorsal midfoot prominence 10 and bracing the foot 2 at the ankle, in accordance with embodiments. The unloader assembly 110 includes a brace 172 formed of a flexible, elastic material that wraps around the foot 2 where it joins the ankle. The brace 172 can provide mechanical support to the foot 2 and ankle. The brace 172 has a distal pocket 170a and a proximal pocket 170b arranged distal and proximal to the dorsal midfoot prominence, respectively. The distal and proximal pockets 170a, 170b retain spacers 122. The spacers 122 project dorsally from the foot 2 distal and proximal to the dorsal midfoot prominence 10. In some embodiments, the brace 172 may include one of the distal or proximal pockets 170a or 170b, rather than both.

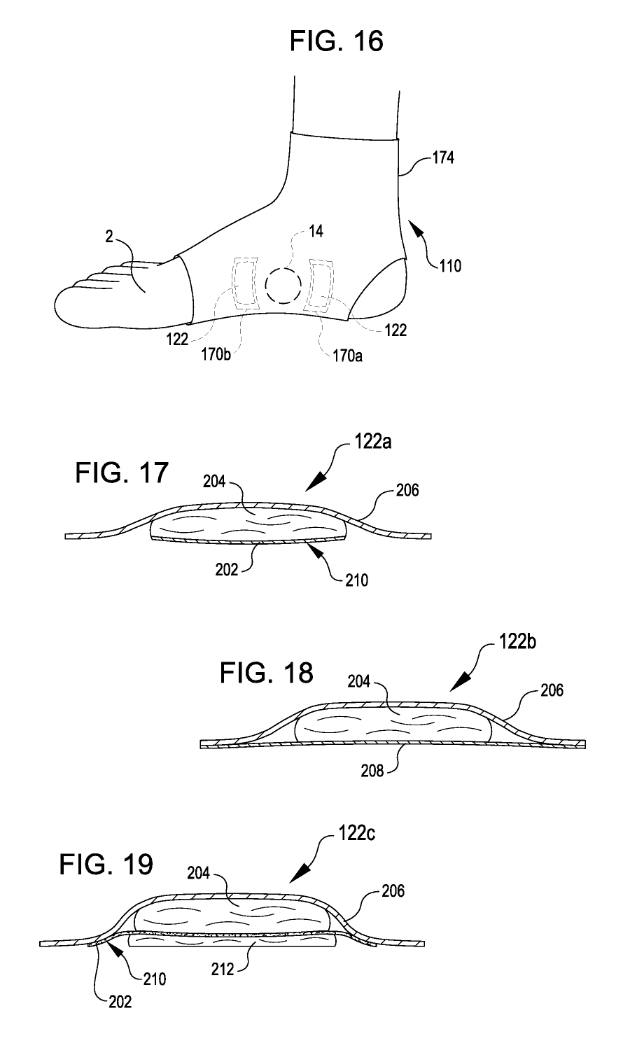

FIG. 16 shows an alternative unloader assembly 110 for unloading pressure on an accessory navicular 14 and bracing the foot 2 at the ankle, in accordance with embodiments. The unloader assembly 110 includes a brace 174 formed of a flexible, elastic material that wraps around the foot 2 where it joins the ankle. The brace 174 can provide mechanical support to the foot 2 and ankle. The brace 174 has a distal pocket 170b and a proximal pocket 170a arranged distal and proximal to the accessory navicular 14, respectively. The distal and proximal pockets 170b, 170a retain spacers 122. The spacers 122 project outward from the foot 2 distal and proximal to the accessory navicular 14. In some embodiments, the ankle brace 172 may include one of the distal or proximal pockets 170b or 170a, rather than both.

FIG. 17 shows a side section view of a spacer 122a, showing a first layering arrangement, in accordance with embodiments. The spacer 122a has a contact layer 202 for contacting the skin of a patient. The contact layer 202 is preferably formed of a biocompatible material, e.g., a biocompatible plastic, rubber, or gel. The contact layer 202 may also be formed of a tacky material for preventing sliding of the spacer 122a. The contact layer 202 may also include a coating 210, which may be a tacky, or biocompatible adhesive coating. The coating 210 may include an antimicrobial agent. A spacing layer 204 is connected with the contact layer 202. The spacing layer 204 is arranged to provide cushioning and spacing above the contact layer 202. The spacing layer 204 may be formed of a cushioning, biocompatible material, such as a soft plastic, gel, rubber, gauze, or other suitable material. The spacing layer 204 is connected with an outer layer 206, which can be formed of a flexible and elastic material. The outer layer 206 can include, or can be connected with, a primary securement for connecting the spacer 122a with a patient.

FIG. 18 shows a side section view of a spacer 122b, showing a second layering arrangement, in accordance with embodiments. The spacer 122b has a spacing layer 204 sandwiched between an inner layer 208 which is arranged for contacting the skin of a patient and an outer layer 206. The spacing layer 204 and outer layer 206 are similar to the spacing and outer layers of FIG. 17. The inner layer 208 can include a flexible, elastic biocompatible material suitable for prolonged contact with human skin. The inner layer 208 may include, for example, a soft and stretchy fabric layer. The inner layer 208 can be connected with the spacing layer 204, and may further be connected with the outer layer 206, e.g., by being adhered, sewn, or otherwise connected with the outer layer. The spacing layer 204 may be removable. Where the spacing layer 204 is removable, inner layer 208 and outer layer 206 can collapse and lie flat. Various spacing layers 204 of varying thicknesses may be provided and may be interchangeably added between the inner and outer layers 208, 206, to facilitate different therapeutic thicknesses of the spacer 122b, e.g., for different sized prominences.

FIG. 19 shows a side section view of a spacer 122c, showing a third layering arrangement, in accordance with embodiments. The spacer 122c has a spacing layer 204, an outer layer 206, and a contact layer 202 with a coating 210, similar to the layers disclosed above with respect to FIGS. 17 and 18. The contact layer 202 can be disposed around a second spacing layer 212, which can supplement the spacing layer 204 by, e.g., providing further cushioning, form fitting, or other effect. For example, the second spacing layer 212 may include an antimicrobial material, an absorbent material, a form-fitting gel, or other suitable material.

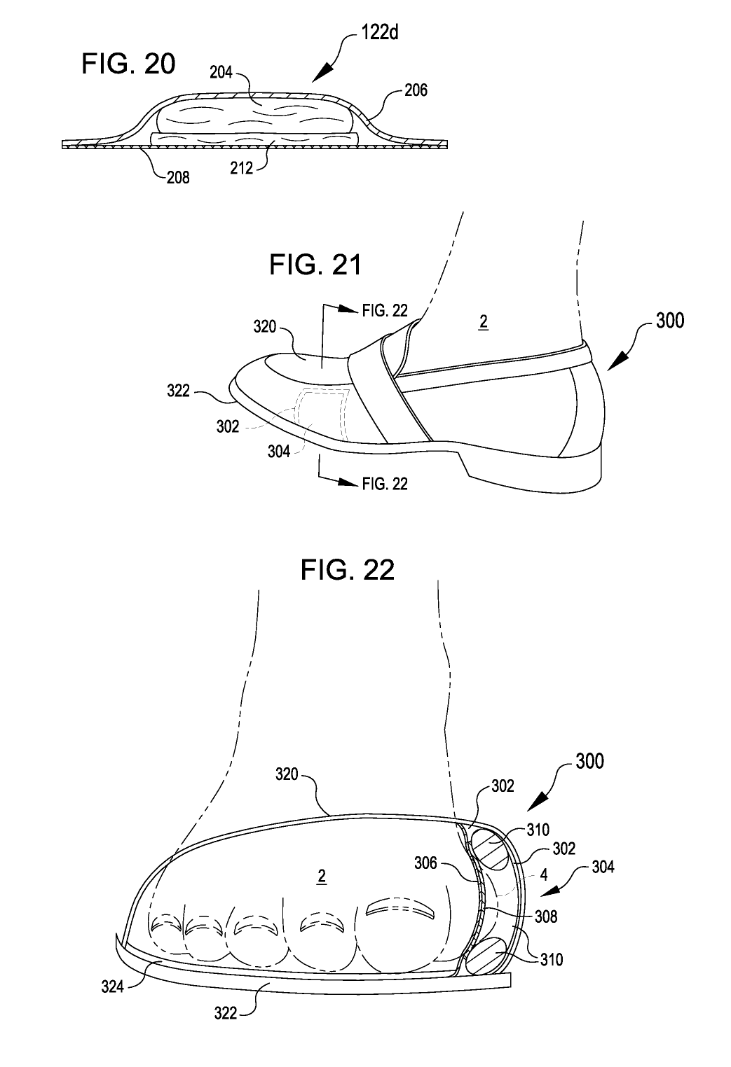

FIG. 20 shows a side section view of a spacer 122d, showing a fourth layering arrangement, in accordance with embodiments. The spacer 122d has a spacing layer 204, outer layer 206, and inner layer 208, similar to layers disclosed above with respect to FIGS. 17 and 18. A second spacing layer 212 can be sandwiched between the inner layer 208 and the spacing layer 204. The second spacing layer 212 can supplement the spacing layer 204 by, e.g., providing a secondary cushioning effect. For example, the second spacing layer 212 may be a form-fitting gel configured to deform around the physiology of a patient, while the first spacing layer 204 provides structure. By way of further example, the second spacing layer 212 may be an absorbent layer, such as a gauze layer, while the first spacing layer 204 provides structure and/or cushion. The second spacing layer 212 may be connected with the first spacing layer 204 at one side and with the inner layer 208 at the other side. In some embodiments, additional layers may be provided in a similar manner with different properties, or some layers may be omitted, e.g., the inner layer 208.

FIG. 21 shows a perspective view of an orthopedic shoe 300 for unloading pressure on a pathological prominence, in accordance with embodiments. The shoe 300 has a sole 322 and a body 320, and a cavity 302 is disposed in the body 320 of the shoe for accommodating the pressure unloading assembly 304. The pressure unloading assembly 304 is provided in the cavity 302 for unloading pressure from the prominence, e.g., a bunion 4 of a patient (FIG. 22). Although presented with reference to a bunion 4, the shoe 300 may be arranged to have a cavity and unloading assembly at any other suitable location for unloading a different pathological prominence, similar to the unloading assemblies described above with reference to FIGS. 1-16.

FIG. 22 shows a side section view of the shoe 300 of FIG. 21. The foot 2 rests inside the body 320 of the shoe 300 above a sole 322 and insole 324. The pathological prominence, i.e. bunion 4, projects from the side of the foot 2 into the cavity 302 of the shoe 300. The pressure unloading assembly 304 is disposed adjacent to the bunion 4. The pressure unloading assembly can include a spacer 310, which may be shaped to rest adjacent to the bunion 4 without pressing on the bunion, and a contact layer 306 for contacting the skin of the foot 2. An additional cushioning layer 308 may be provided around the bunion for improving the comfort of a user. The cushioning layer 308 and/or contact layer 306 may include absorbent and/or antimicrobial materials.

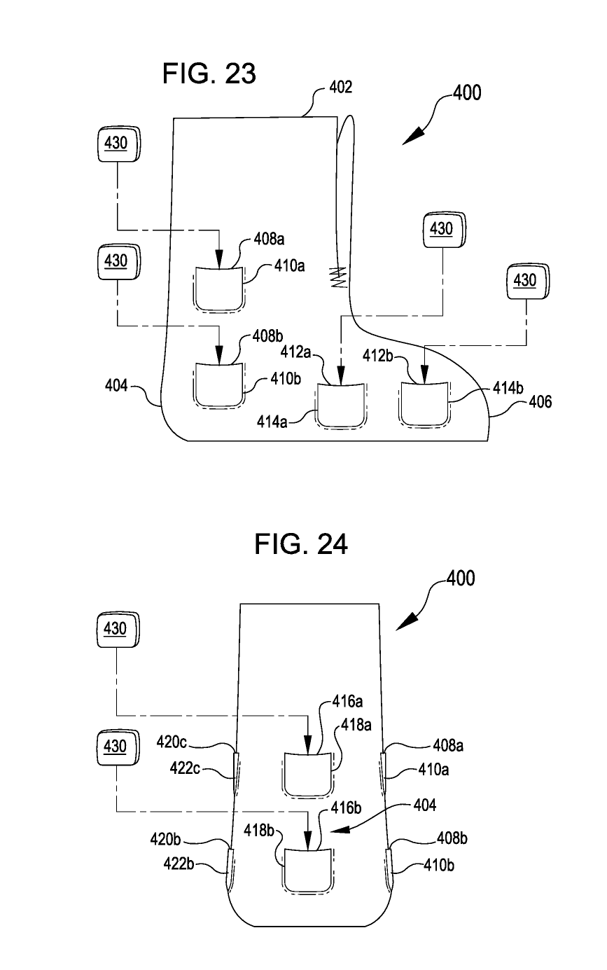

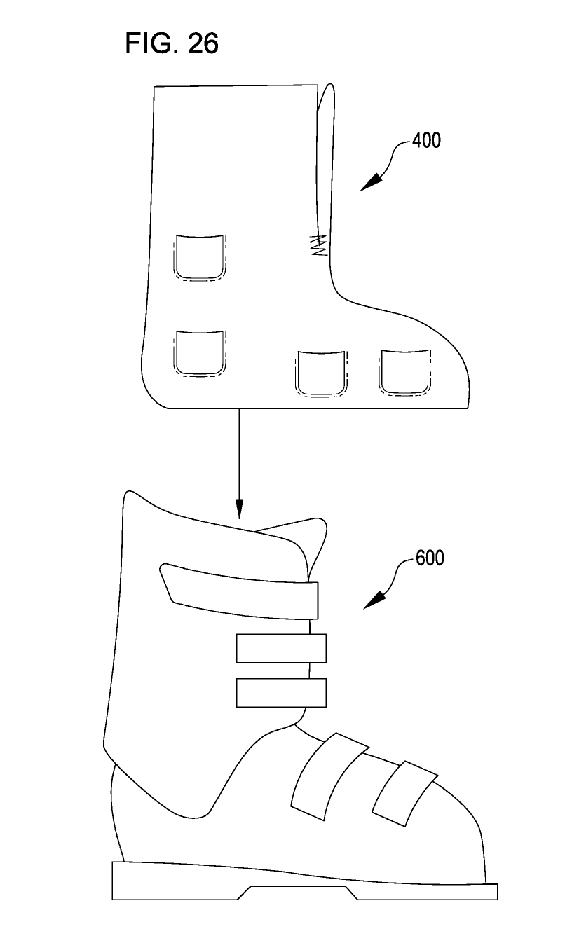

FIG. 23-26 show pressure unloading liners 400, 500 for use in footwear, in accordance with embodiments. The liner 400, 500 is sized to fit within footwear between a foot of a user and an outer shell of the footwear. According to some embodiments, the liner 400, 500 can be a cushioned and/or insulating layer that protects the user's foot inside an outer shoe, boot, or comparable article of footwear. In specific embodiments, the liner is sized and configured for insertion into a sports shoe or a sports boot, such as a hiking boot, mountaineering boot, or a boot for snow sports such as skiing, snowboarding, and cross-country or the like. In some other embodiments, the liner can be sized to fit in a conventional shoe in order to provide padding or insulation around the foot. In some preferred embodiments, the liner is a soft foam or comparable, insulating material for protecting a foot in a hard-shelled outer boot, such as a downhill ski boot, hard-shelled mountaineering boot, cross-country ski boot, or similar boot, as shown in FIG. 26.

FIG. 23 shows a pressure unloading liner 400 in a side view (showing a medial side, i.e. an inner side of a left liner) in accordance with embodiments. The liner 400 is sized for accommodating a foot of a user, and for fitting into an outer shell, shoe, or boot. The liner 400 is formed of a liner body 402 having a toe end 406 and a heel end 404. The liner body 402 includes a series of pockets 414a, 414b, 410a, 410b in the liner body that are capable of holding spacers 430 therein. The spacers 430 are similar to spacers described above, e.g., spacer 122 shown in FIG. 1. Each pocket (410a, 410b, 414a, 414b) is accessible via a respective slit in the liner body 402 (e.g., elongate slits 408a, 408b, 412a, 412b) that allow for insertion of a respective spacer 430 therein.

Each pocket is positioned along the liner 400 adjacent to a position of a bony prominence. For example, pockets 414a, 414b near the toe end 406 can be positioned posterior to and anterior to a position of a bunion (i.e., laterally adjacent to the bunion, as opposed to directly adjacent to the bunion). Similarly, pockets 410a, 410b are positioned above and below a position of a medial malleolus. Pockets may be positioned in the pressure unloading liner 400 at any suitable position or pairs of positions that are laterally adjacent to any suitable bony prominence of the foot or ankle, or as described above. For example, FIG. 24 shows the pressure unloading boot liner 400 of FIG. 23 in a rear view, showing pockets 418a, 418b positioned above and below a heel position at the heel end 404 of the liner. Pockets 418a, 418b are similarly accessible via elongate slits 416a, 416b for inserting spacers 430 therein. Similarly, pockets 422a, 422b are shown positioned above and below a location associated with a lateral malleolus, in accordance with embodiments. Pockets 422a, 422b are accessible for insertion of spacers 430 via elongate slots 420a, 420b.

Any of the aforementioned pockets or pairs of pockets can be used in conjunction with one or more spacers to increase an effective thickness of the liner 400 at positions adjacent to one or more problematic bony prominences of the foot, for example, when a bony prominence has become painful or pathological (as in a bunion, a “pump bump,” an inflamed lateral or medial malleolus, or the like). By inserting one or more spacers 430 adjacent to the painful or pathological bony prominence, a user can unload the targeted prominence when the liner is inserted in the shoe or boot, resulting in improved comfort and recovery.

The pressure unloading liner 400 of FIGS. 23-24 provides a user with the capability of unloading a bony prominence of the foot by loading a laterally adjacent part of the foot to press the liner away from the bony prominence. In alternative embodiments, a liner can operate instead, or in addition, by allowing a user to effectively reduce a thickness of a liner directly adjacent, or on top of, a painful or pathological prominence by removing material from the liner.

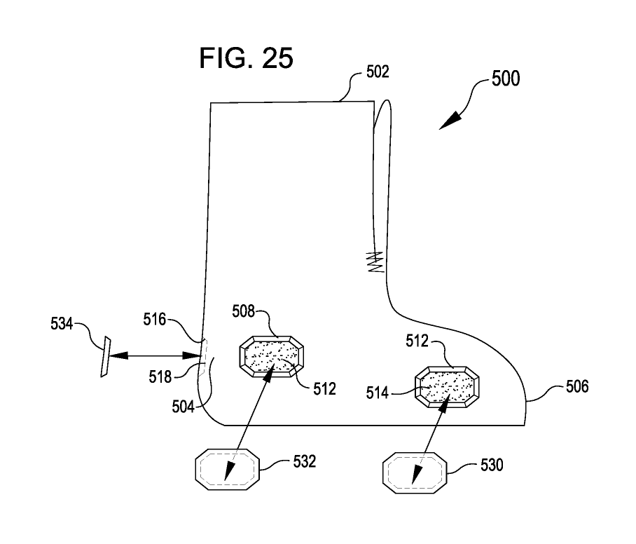

FIG. 25 shows a side view of a second pressure unloading boot liner 500 having voids 508, 512, 516 positioned directly adjacent to locations of bony prominences, with removable spacers 530, 532, 534 therein, in accordance with embodiments. Liner 500 is formed of a liner body 502, with a toe end 506 and heel end 504, similar to liner 400 (FIG. 23). A medial side of the left liner is shown. A first void 512 is positioned directly adjacent a position of a bunion. A second void 508 is shown directly adjacent a position of a medial malleolus. A third void 516 is shown directly adjacent a position of the back of a heel (as implicated in a “pump bump,” or Haglund's deformity). Each respective void 508, 512, 516 is sufficiently deep to thin the material of the liner body 502 when no spacer is present therein. Spacers 530 can be attached with each void 508, 512, 516 to fill in any respective void that is not needed to meet the needs of a particular user. Each void 508, 512, 516 can include attachment features 510, 514, 518 for attaching a spacer 530 therein. In some cases, the attachment features 510, 514, 518 can be reusable attachment features such as VELCRO loop material by Velcro Industries B.V., pile for attachment thereto, an adhesive layer, a hook, strap, or pouch, a fitted cavity into which the spacers 530 can be inserted, or the like. In some alternative embodiments, voids 508, 512, 516 can be covered by a thin layer of material, and spacers 530 can be inserted therein through slits or comparable openings, similar to the pockets described above with reference to FIGS. 23-24.

FIG. 26 shows a side view of a sports boot 600 receiving the pressure unloading liner 400 of FIGS. 23-24. In various embodiments, the liner 400 is removable from the sports boot 600, such that a user can readily modify the liner 400 prior to inserting the liner into the sports boot. It will be understood that liner 400 can also be permanently inserted into the boot 600 after modification and fixed therein, e.g. by way of an adhesive or permanent connector. Similarly, a boot liner like liner 500 (FIG. 25) could also be inserted into the sports boot 600, and could be modified or fixed therein in similar manner to the liner 400 (FIGS. 23-24). The sports boot 600 may be any suitable hard-shelled outer boot, such as a downhill ski boot, hard-shelled mountaineering boot, cross-country ski boot, or similar boot.

The liners 400, 500 shown in FIGS. 22-26 are configured to receive spacers (e.g., 430, 530) into voids or pockets from an exterior of the liner. However, it will be understood that liners 400, 500 can be inverted, and instead receive spacers to voids or pockets interior to the liner and adjacent to a foot of a user. Spacers 430, 530, 532, 534 described above with reference to FIGS. 22-25 can be made of the same or similar materials as the spacers described above with reference to FIGS. 1-21, e.g., cushioning material, such as a soft plastic, gel, rubber, gauze, or other suitable material.

In alternative embodiments, spacers may be customized to match the physiology of a particular patient. For example, spacers may be constructed at various heights to accommodate pathological prominences of different severities. By way of further example, spacers may be constructed of stiffer, or softer, materials to accommodate patient comfort and preference. Spacers may be molded, e.g., in a thermoset or chemically set material, to conform to a shape of the patient's physiology. In some cases, spacers may be customized based on a patient's physiology, e.g., by 3D printing or casting. Removable spacers may be provided at different heights, such that a patient can use different spacers for different physical applications. For example, narrower and/or softer spacers may be provided for everyday use, and thicker and/or more rigid spacers may be provided for active use.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A pressure unloading device, comprising:
a securement shaped to wrap around a part of a foot of a human; and
a spacer connected with the securement and configured to rest proximate to a pathological prominence of the foot, the prominence having a first height;
wherein the spacer has a second height, the second height substantially equal to or greater than the first height; and
wherein the securement is configured to secure the spacer on a surface of the foot proximate to the pathological prominence.

2. The device of claim 1, wherein the spacer is shaped to abut and partially accommodate a portion of the prominence.

3. The device of claim 1, wherein the securement is configured to secure the spacer at a position on the surface of the foot that is laterally adjacent to the pathological prominence.

4. The device of claim 1, wherein:
the spacer is a first spacer, and further comprising:
a second spacer, wherein:
the securement is configured to secure the second spacer on the surface of the foot, proximate to the pathological prominence and on an opposite side of the pathological prominence from the first spacer.

5. The device of claim 1, further comprising one or more secondary securement straps, the secondary securement straps configured to prevent rotation of the securement around the foot.

6. The device of claim 1, wherein:
the pathological prominence is a bunion; and
the spacer is positioned on the securement to rest laterally adjacent the bunion when the securement is placed on the foot.

7. The device of claim 1, wherein:
the pathological prominence is a Taylor's bunion; and
the spacer is positioned on the securement to rest laterally adjacent the Taylor's bunion when the securement is placed on the foot.

8. The device of claim 1, wherein:
the pathological prominence is a medial malleolus;
the spacer is positioned on the securement to rest laterally adjacent the medial malleolus when the securement is placed on the foot.

9. The device of claim 1, wherein:
the pathological prominence is a ganglion; and
the spacer is positioned on the securement to rest laterally adjacent the ganglion when the securement is placed on the foot.

10. The device of claim 1, wherein:
the pathological prominence is a “pump bump;” and
the spacer is positioned on the securement to rest laterally adjacent the “pump bump” when the securement is placed on the foot.

11. The device of claim 1, wherein:
the pathological prominence is a lateral malleolus; and
the spacer is positioned on the securement to rest laterally adjacent the lateral malleolus when the securement is placed on the foot.

12. The device of claim 1, wherein:
the securement further comprises a brace configured to provide support to the foot.

13. A pressure unloading device, comprising:
a shoe;
a spacer integrally connected with the shoe and configured to rest proximate to a pathological prominence of a foot of a human when the shoe is worn, the prominence having a first height; wherein
the spacer has a second height, the second height substantially equal to or greater than the first height; and
the shoe comprises a void adjacent to the spacer and around the prominence.

14. A pressure unloading liner for footwear, comprising:
a liner body shaped to hold a foot and to fit within a boot while separating the foot from the boot;
a pocket in the liner, the pocket positioned based on a location of a bony prominence in the foot, the prominence having a first height;
a spacer sized to fit in the pocket, the spacer having a second height substantially equal to or greater than first height.

15. The liner of claim 14, further comprising:
an elongate slit in a surface of the liner body; wherein
the pocket comprises a void in a material of the liner body accessible from the elongate slit; and
the pocket is sized to receive the spacer, such that the spacer is positioned laterally adjacent the bony prominence along the foot when the spacer is received in the pocket.

16. The liner of claim 15, wherein the pocket is a first pocket, and further comprising:
a second elongate slit and a second pocket accessible from the second elongate slit; wherein:
the second pocket is positioned laterally adjacent to the bony prominence and opposite the first pocket relative to the bony prominence.

17. The liner of claim 14, further comprising:
a plurality of pockets in the liner, each pocket of the plurality of pockets being positioned laterally adjacent at least one of a respective plurality of bony prominences in the foot;
a plurality of elongate slits in the liner, each elongate slit opening into a respective pocket of the plurality of pockets.

18. The liner of claim 14, wherein:
the pocket comprises a void in a material of the liner body;
the pocket is positioned directly adjacent to the bony prominence such that the bony prominence presses the material of the liner body into the void when the liner is worn and when the spacer is removed from the pocket.

19. The liner of claim 14, further comprising:
a plurality of pockets in the liner, each pocket of the plurality of pockets being positioned directly adjacent to a respective bony prominence of a plurality of bony prominences in the foot; and
a plurality of spacers, each sized to fit in a respective pocket of the plurality of pockets, each spacer having a height suitable to unload pressure from a respective bony prominence associated with the respective pocket when removed from the pocket.

20. A sports boot, comprising:
the liner of claim 14; and
an outer shell sized to receive the liner.
